(19) United States
(12) Patent Application Publication
(10) Pub. No.: US 2010/0107319 A1
(43) Pub. Date: May 6, 2010

(54) One-Sided Transparent Film For Ski Goggles

(76) Inventor: Samuel Yudkin, Bellaire, TX (US)

Correspondence Address: KING & SPALDING, LLP, 1100 LOUISIANA ST., STE. 4000, ATTN.: IP Docketing, HOUSTON, TX, 77002-5213, US

(21) Appl. No.: 12/262,045

(22) Filed: Oct. 30, 2008

(51) Int. Cl. A61F 9/02; A41D 27/08

(52) U.S. Cl. 2/426; 2/244

(57) ABSTRACT

A ski goggle having a one-sided transparent film applied to the lens and the method of using the one-sided transparent film. The one-sided transparent film comprises a transparent side, a non-transparent side, and a design, which may include, but is not limited to, a logo, slogan, advertisement, words or a statement. The one-sided transparent film is coupled to the lens of the ski goggle, wherein the transparent side faces the same direction as the interior side of the lens and the non-transparent side faces the same direction as the exterior side of the lens. This design may be distinctly viewed from the exterior side of the lens, but not from the interior side of the lens; thereby not impairing the user's vision.

### CROSS REFERENCE TO RELATED APPLICATIONS

The present application claims priority from U.S. Provisional Patent Application No. 60/983,782, entitled “One-sided Transparent film For Ski Goggles and Other Eyewear” and filed on Oct. 30, 2007.

### BACKGROUND OF THE INVENTION

This invention relates generally to ski goggles and, more particularly, to lenses for ski goggles.

### BRIEF DESCRIPTION OF THE DRAWINGS

The foregoing and other features and aspects of the invention will be best understood with reference to the following description of certain exemplary embodiments of the invention, when read in conjunction with the accompanying drawings, wherein:

FIG. 1 is a perspective view of a ski goggle in accordance with the prior art;

FIG. 2 is a perspective view of a ski goggle comprising at least one lens having a one-sided transparent film as viewed from the exterior side of the lens in accordance with an exemplary embodiment;

FIG. 3 is a perspective view of a ski goggle comprising at least one lens having a one-sided transparent film as viewed from the exterior side of the lens in accordance with another exemplary embodiment;

FIG. 4 is a perspective view of the exterior side of a ski goggle as worn by a user in accordance with an exemplary embodiment;

FIG. 5 is a perspective view of a ski goggle comprising at least one lens having a one-sided transparent film as viewed from the interior side of the lens in accordance with an exemplary embodiment;

FIG. 6 is a perspective view of a ski goggle comprising at least one lens having a one-sided transparent film as viewed from the exterior side of the lens in accordance with another exemplary embodiment; and

FIG. 7 is a perspective view of a ski goggle comprising at least one lens having a one-sided transparent film as viewed from the exterior side of the lens in accordance with yet another exemplary embodiment.

### DETAILED DESCRIPTION OF THE INVENTION

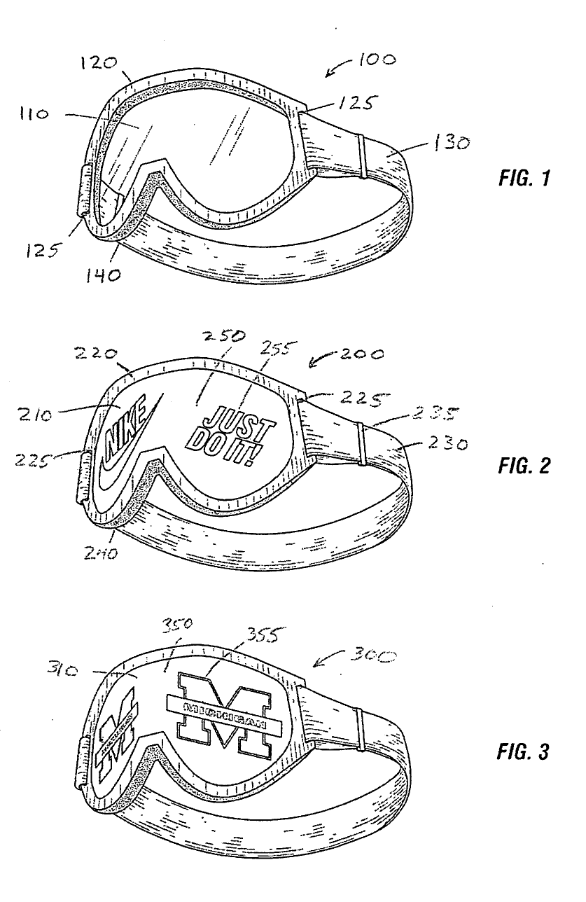

FIG. 1 is a perspective view of a ski goggle 100 in accordance with the prior art. FIG. I illustrates a prior art ski goggle 100 consisting a lens 110, a frame 120 having an eyelet 125 on each side of the frame 120, and a band 130 attached to the two eyelets 125. The ski goggle 100 may also include a seal 140, as shown in FIG. 1, surrounding the perimeter of the frame 120. The seal 140 may include a spongy material that allows the ski goggle 100 to fit more comfortably on a user's head. The lens 110 is attached to the frame 120 according to known methods in the art. The lens 110 is clear and transparent when viewed from both the interior and exterior sides of the lens 110. The lens 110 may be made of polycarbonate material or glass.

FIG. 2 is a perspective view of a ski goggle 200 comprising at least one lens 210 having a one-sided transparent film 250 as viewed from the exterior side of the lens 210 in accordance with an exemplary embodiment. FIG. 2 illustrates a ski goggle 200 comprising at least one lens 210, a frame 220 having a connector 225 on each side of the frame 220, a band 230 attached to the two connectors 225, and at least one one-sided transparent film 250 coupled to the at least one lens 210. The ski goggle 200 may also include a seal 240, as shown in FIG. 2, surrounding the perimeter of the frame 220.

The lens 210 may be coupled to the frame 220 using an adhesive on the interior side of the perimeter of the frame 220 so that the lens 210 may be coupled to the interior side of the frame's 220 perimeter. Alternatively, the lens 210 may be coupled to the frame 220 using an adhesive on the exterior side of the frame 220 so that the lens 210 may be coupled to the exterior side of the frame's 220 perimeter. Furthermore, the lens 210 may be coupled to the frame 220 using a groove (not shown) located within at least a portion of the interior edge of the frame 220 so that at least a portion of the perimeter of the lens 210 is coupled to the portion of the frame's 220 grooved edge. Although the coupling of the lens 210 to the frame 220 has been illustrated using an adhesive or a groove, other coupling methods known to those of ordinary skill in the art may be used without departing from the scope and spirit of the exemplary embodiment.

The lens 210 may be either clear or tinted, but is transparent when viewed from either the interior or exterior sides of the lens 210. The lens 210 may be made of polycarbonate material or glass. Although this embodiment uses a lens 210 made of polycarbonate material or glass, other materials that are transparent when viewed from either the interior or exterior sides of the lens 210 may be used without departing from the scope and spirit of the exemplary embodiment.

Although this embodiment shows a single lens 210 coupled to the frame 220, the frame 220 may be designed such that there may be more than one lens 210 coupled to the frame 220 without departing from the scope and spirit of the exemplary embodiment. For example, instead of a single lens covering both eyes of a user, two separate lenses may be used, wherein each lens covers only one eye of a user, without departing from the scope and spirit of the exemplary embodiment.

The frame 220 comprises a connector 225 on each side of the frame 220. The connector 225 may assist in coupling a band 230 to the frame 220. The connector 225 may be an eyelet, as shown in FIG. 2. Although this embodiment uses an eyelet as the connector 225, other types of connectors 225 may be used, including but not limited to a hook or a screw, without departing from the scope and spirit of the exemplary embodiment. The frame 220 may be made of metal, plastic, polycarbonate material, glass, other polymers or any combination of these materials. Although this embodiment discloses the frame 220 as being made from metal, plastic, polycarbonate material, glass, other polymers or any combination of these materials, other suitable materials known to those of ordinary skill in the art may be used without departing from the scope and spirit of the exemplary embodiment.

The band 230 may be attached to the two connectors 225 so that the ski goggle 200 is securely attached to the user's head. The band 230 may be made from a stretchable material, much like a rubber band, so that it securely couples the ski goggle 200 to the user's head. In an alternative embodiment, the band 230 may be made from a non-flexible material, but may include an adjuster 235 for adjusting the length of the band 230 according to the circumference of the user's head. In another embodiment, the band 230 may be made from a flexible material and may also include an adjuster 235 for adjusting the length of the band 230 according to the circumference of the user's head. In yet another embodiment, there may be no band 230 or connector 225 since the ski goggle 200 may be custom-made for the user's face. In this embodiment, the shape of the ski goggle 200 conforms to the user's face such that the shape provides a secure coupling of the ski goggle 200 to the user's face.

The one-sided transparent film 250 may be fabricated from a film or a plastic lens gel. This one-sided transparent film 250 may be transparent when viewed from one side of the one-sided transparent film 250, but may not be transparent when viewed from the opposite side of the one-sided transparent film 250. Additionally, the one-sided transparent film 250 comprises a design 255 which may be distinctly viewed from only the non-transparent side of the one-sided transparent film 250. According to some embodiments, the design 255 may comprise a hologram, which may be distinctly seen only from the non-transparent side of the one-sided transparent film 250. The one-sided transparent film 250 comprises an adhesive on one side so that it may be coupled to the lens 210. Depending upon the side the adhesive is located, the one-sided transparent film 250 may be coupled to the interior side of the lens 210; or alternatively, the one-sided transparent film 250 may be coupled to the exterior side of the lens 210. In one example, the adhesive may be coupled to the transparent side of the one-sided transparent film 250 so that the one-sided transparent film 250 may be coupled to the exterior side of the lens 210. In another example, the adhesive may be coupled to the non-transparent side of the one-sided transparent film 250 so that the one-sided transparent film 250 may be coupled to the interior side of the lens 210. Regardless, the non-transparent side of the one-sided transparent film 250 should be facing the exterior side of the lens 210 once the one-sided transparent film 250 is coupled to the lens 210 so that the user's vision is not impaired. In an exemplary embodiment, the design 255 may be a color which contrasts the color of the remaining lens 210. For example, the design 255 may be gold in color, while the remaining lens 210 surrounding the design 255 is darker in color. Although an exemplary color has been illustrated for the design 255 and the remaining lens 210 surrounding the design 255, alternative color combinations may be used without departing from the scope and spirit of the exemplary embodiment.

The design 255 on the one-sided transparent film 250 may be anything the user wishes to promote, such as an advertisement for marketing a service or product, or be associated with. The design 255 may include at least one logo, at least one word, or a combination of at least one logo and at least one word. Examples of designs 255 include, but are not limited to, logos, slogans, pictures, advertisements, characters, and statements. Some examples of logos include, but are not limited to, company logos, university logos, and team logos. Some examples of slogans include, but are not limited to, company slogans, university slogans, and team slogans. Some examples of advertisements include, but are not limited to, company advertisements, university advertisements, and team advertisements. Some examples of statements include, but are not limited to, personal statements and/or personal advertisements. In the embodiment shown in FIG. 2, a company logo and slogan are used as the design 255.

The seal 240 may include a spongy material that allows the ski goggle 200 to fit more comfortably on a user's face. Alternatively, the seal 240 may include a gel material. This seal 240 may be coupled to the perimeter of the ski goggle's 200 frame 220. Although a spongy material or a gel material may be used for the seal 240, any material may be used, including but not limited to foam, rubber or any other polymer, for providing a comfort fit without departing from the scope and spirit of an exemplary embodiment.

The ski goggle of the exemplary embodiment may be designed for males, females, children and adults.

FIG. 3 is a perspective view of a ski goggle 300 comprising at least one lens 310 having a one-sided transparent film 350 as viewed from the exterior side of the lens 310 in accordance with another exemplary embodiment. The ski goggle 300 has been described above in detail with reference to FIG. 2. In this embodiment, however, a university logo is used as the design 355. Although this embodiment uses a university logo as the design 355, any design may be used without departing from the scope and spirit of the exemplary embodiment.

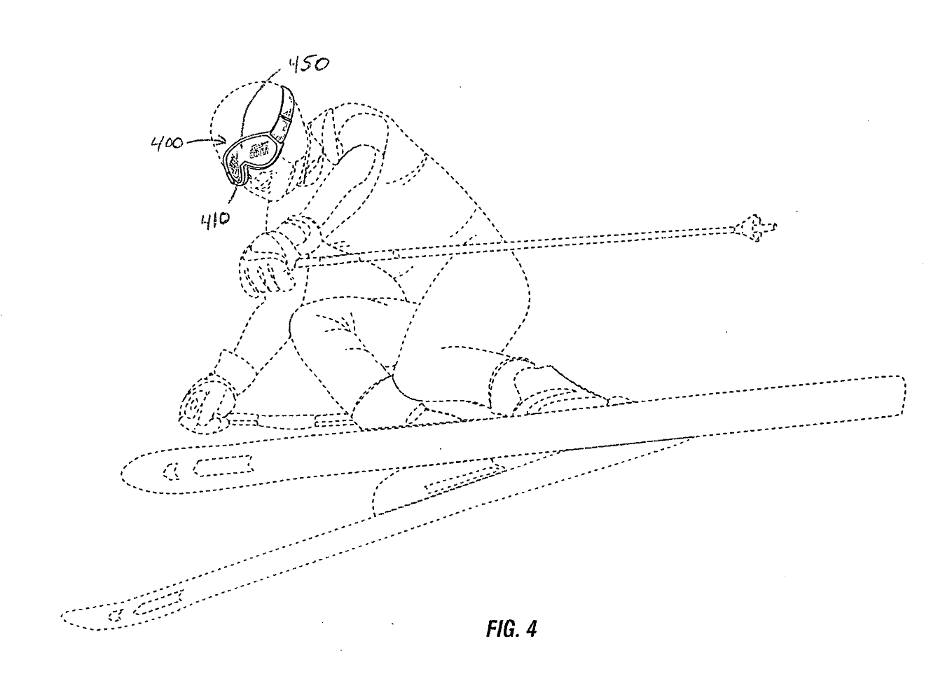

FIG. 4 is a perspective view of the exterior side of a ski goggle 400 as worn by a user in accordance with an exemplary embodiment. The ski goggle 400 of FIG. 2 is securely coupled to the user's face, such that lens of the ski goggle is located substantially in front of the eyes of the user. It may be seen that the non-transparent side of the one-sided transparent film 450 is facing the exterior side of the lens 410 and does not impair the user's vision.

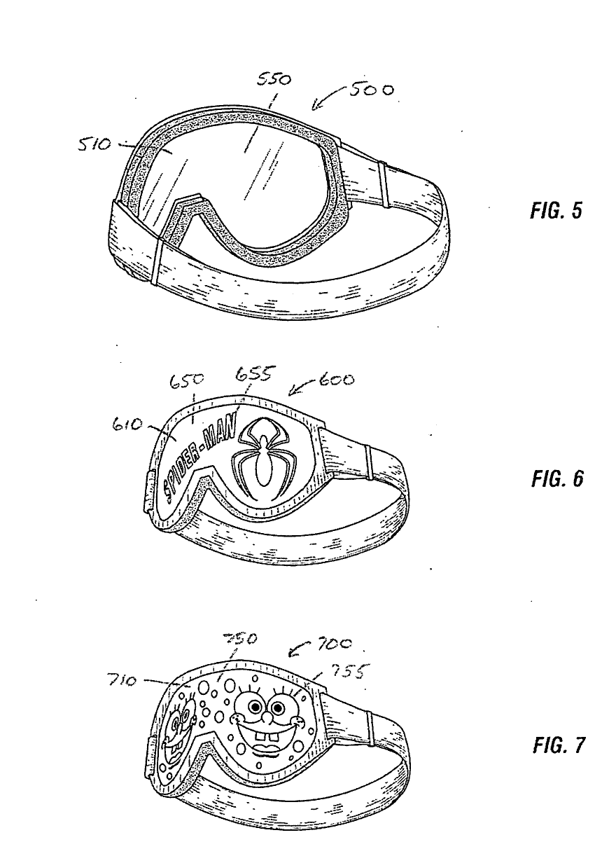

FIG. 5 is a perspective view of a ski goggle 500 comprising at least one lens 510 having a one-sided transparent film 550 as viewed from the interior side of the lens 510 in accordance with an exemplary embodiment. It may be seen that the transparent side of the one-sided transparent film 550 is facing the interior side of the lens 510. On the exterior side (not shown) of the lens 510 is a design (not shown) that may be viewed only from this exterior side. Once the ski goggle 500 is securely coupled to the user's head, the user may not be able to distinctly view the design (not shown) from the interior side of the ski goggle 500. The user's vision may be substantially the same as if there were no one-sided transparent film 550 applied to the lens 510; or, the user may be able to vaguely see an outline of the design, but not enough to impair the user's vision.

FIG. 6 is a perspective view of a ski goggle 600 comprising at least one lens 610 having a one-sided transparent film 650 as viewed from the exterior side of the lens 610 in accordance with another exemplary embodiment. The ski goggle 600 has been described above in detail with reference to FIG. 2. In this embodiment, however, a cartoon logo is used as the design 655. Although this embodiment uses a cartoon logo as the design 655, any design may be used without departing from the scope and spirit of the exemplary embodiment.

FIG. 7 is a perspective view of a ski goggle 700 comprising at least one lens 710 having a one-sided transparent film 750 as viewed from the exterior side of the lens 710 in accordance with yet another exemplary embodiment. The ski goggle 700 has been described above in detail with reference to FIG. 2. In this embodiment, however, a cartoon character is used as the design 755. Although this embodiment uses a cartoon character as the design 755, any design may be used without departing from the scope and spirit of the exemplary embodiment.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims. It is therefore, contemplated that the claims will cover any such modifications or embodiments that fall within the scope of the invention.

What is claimed is:

1. A ski goggle comprising:
a frame of the ski goggle;
at least one lens coupled to the frame, wherein the at least one lens comprises an interior side and an exterior side;
a one-sided transparent film coupled to the at least one lens, wherein the one-sided transparent film comprises a transparent side facing the same direction as the interior side of the at least one lens and a non-transparent side facing the same direction as the exterior side of the at least one lens; and
a design coupled to the one-sided transparent film, wherein the design does not impair the vision of a user when viewing from the interior side of the at least one lens, and wherein the design is distinctly visible when viewing from the exterior side of the at least one lens.

2. The ski goggle of claim 1, wherein the one-sided transparent film comprises a plastic lens gel.

3. The ski goggle of claim 1, wherein the one-sided transparent film comprises a film.

4. The ski goggle of claim 1, wherein the one-sided transparent film comprises an adhesive coupled to the transparent side of the one-sided transparent film.

5. The ski goggle of claim 4, wherein the one-sided transparent film is coupled to the exterior side of the at least one lens via the adhesive.

6. The ski goggle of claim 1, wherein the one-sided transparent film comprises an adhesive coupled to the non-transparent side of the one-sided transparent film.

7. The ski goggle of claim 6, wherein the one-sided transparent film is coupled to the interior side of the at least one lens via the adhesive.

8. The ski goggle of claim 1, wherein the design comprises a hologram.

9. The ski goggle of claim 1, wherein the design is selected from at least one of pictures, advertisements, logos, slogans, statements, and characters.

10. A method for using a one-sided transparent film comprising:
providing a ski goggle comprising a frame and at least one lens coupled to the frame, wherein the at least one lens comprises an interior side and an exterior side;
providing a one-sided transparent film comprising a design, wherein the one-sided transparent film comprises a transparent side and a non-transparent side;
coupling the one-sided transparent film to the at least one lens of the ski goggle, wherein the transparent side of the one-sided transparent film faces the same direction as the interior side of the at least one lens and the non-transparent side of the one-sided transparent film faces the same direction as the exterior side of the at least one lens; and
placing the ski goggle on the face of a user, wherein the at least one lens of the ski goggle is located substantially in front of the eyes of the user,
wherein the design does not impair the vision of the user when viewing from the interior side of the at least one lens, and wherein the design is distinctly visible when viewing from the exterior side of the at least one lens.

11. The method of claim 10, wherein the one-sided transparent film comprises a plastic lens gel.

12. The method of claim 10, wherein the one-sided transparent film comprises a film.

13. The method of claim 10, wherein the one-sided transparent film comprises an adhesive coupled to the transparent side of the one-sided transparent film.

14. The method of claim 13, wherein the one-sided transparent film is coupled to the exterior side of the at least one lens via the adhesive.

15. The method of claim 10, wherein the one-sided transparent film comprises an adhesive coupled to the non-transparent side of the one-sided transparent film.

16. The method of claim 15, wherein the one-sided transparent film is coupled to the interior side of the at least one lens via the adhesive.

17. The method of claim 10, wherein the design comprises a hologram.

18. The method of claim 10, wherein the design is selected from at least one of pictures, advertisements, logos, slogans, statements, and characters.
